(19) United States
(12) Patent Application Publication
(10) Pub. No.: US 2010/0247555 A1
(43) Pub. Date: Sep. 30, 2010

(54) REVERSIBLY INHIBITED ANTIBODIES FOR IMMUNE CELL STIMULATION

(75) Inventors: Colin Henry Self, Northumberland (GB); Stephen Thompson, Northumberland (GB)

Correspondence Address: DANN, DORFMAN, HERRELL & SKILLMAN, 1601 MARKET STREET, SUITE 2400, PHILADELPHIA, PA, 19103-2307, US

(73) Assignee: BIOTRANSFORMATIONS LIMITED, Northumberland (GB)

(21) Appl. No.: 12/293,747

(22) PCT Filed: Mar. 21, 2007
(86) PCT No.: PCT/GB2007/001023

§ 371 Date: Jun. 15, 2010

(30) Foreign Application Priority Data
Mar. 21, 2006 (GB) 0605702.0

(51) Int. Cl. A61K 39/395; C07K 16/28; A61P 31/00

(52) U.S. Cl. 424/173.1; 530/389.6

(57) ABSTRACT

The invention provides methods for stimulating the immune system using antibodies capable of activating immune cells, such as T cells, the antibodies having reversibly inhibited antigen binding sites. Selective activation of the antigen binding sites at a site where an immune response is required (e.g. by irradiation) leads to a corresponding activation of the immune response at that site. Surprisingly good results are achieved even though the antibodies do not possess any targeting moiety which causes them to selectively accumulate at the site where immune cell activation is required.

### FIELD OF THE INVENTION

The invention relates to methods for stimulating the immune response in a specific manner at defined regions of the body, for example at the site of a tumour.

### BACKGROUND TO THE INVENTION

A key clinical objective is to reduce the side-effects of therapeutic agents by making them as specific as possible. The therapeutic monoclonal antibody industry has been built on the promise of being able to harness the exquisite specificity of the immune system. It is clear, however, that achieving the desired level of specificity, in the complex milieu of the human body, still poses a substantial challenge.

It has been suggested to target the immune response to a tumour using bispecific antibodies¹⁻⁴. In theory one part of the antibody reacts with a specific tumour antigen and binds to the tumour cell surface whilst the other part of the antibody reacts with a T-cell marker (normally CD3) thus targeting the T-cell to the tumour cell. In practice this procedure suffers from two major drawbacks.

Firstly, it has proved to be very difficult to obtain the specificity required for antibodies to differentiate between tumour and normal cells⁵, perhaps not surprising when the large ratio of normal tissue to tumour tissue in most patients is taken into consideration. This limits the degree of specific localisation that can be achieved against most tumours. Currently only a very few antibodies have been licensed for use against solid tumours⁶. Secondly, the introduction of anti-T-cell based bispecific constructs results in them being bound by peripheral T-cells before the bispecific antibody reaches its tumour target. This both impedes the bispecific antibody from reaching the tumour and activates T-cells peripherally, leading to T-cell depletion and unwanted cytokine storms^{1,4,7}.

WO96/34892 proposes that the anti-CD3 half of the bispecific antibody should be reversibly inhibited or inactivated. The anti-tumour portion of the antibody remains free to circulate and bind to tumour cells, but the anti-CD3 portion should not be able to bind, activate and remove peripheral T-cells from the patient's circulation. Non-specific cross reactions or specific unwanted binding of the anti-tumour antibody should become irrelevant as the anti-CD3 portion of the antibody would be inactive until it was reactivated in the required region. A further advantage would be that higher doses of conjugate could be administered, providing more conjugate to target the tumour.

### SUMMARY OF THE INVENTION

The present inventors have investigated the therapeutic potential of reversibly inhibited antibodies capable of activating immune cells. It has been found that such antibodies, when re-activated, are surprisingly effective at stimulating localised immune responses even though the antibodies themselves do not carry binding functionalities which specifically target them to the region of the body where the immune response is required. In the Examples, it is shown that T cell-activating antibodies which are inactivated by photocleavable moieties are capable of stimulating a significant anti-tumour response when their binding activity is restored by irradiation, despite the absence of any targeting moiety which causes the antibody to be localised to the tumour.

In a first aspect, the present invention provides the use of an antibody in the preparation of a medicament for stimulating an immune response,

the antibody comprising an antigen binding site capable of binding to a cell of the immune system wherein binding of said antibody to said cell of the immune system activates said cell of the immune system,
wherein said antigen binding site is reversibly inhibited from binding to said cell of the immune system,
and wherein the antibody does not comprise a targeting moiety capable of binding to a cell which is not said cell of the immune system.

The antibody can be administered to a subject and selectively activated at a given physiological site where an immune response is desired, by restoring the ability of the antigen binding site to bind its cognate epitope on the cell of the immune system. The antibody is then able to bind to, and activate, the cell of the immune system.

The cell may be any cell of the immune system whose activation, at the required physiological site, can stimulate or enhance an immune response there. Suitable cells include T cells, including αβ T cells and γδ T cells.

The cell may be a cytotoxic T cell. Activation of cytotoxic T cells is particularly appropriate when it is desirable to stimulate an immune response against one of the subject's own cells, e.g. a parasitised cell (e.g. one carrying a virus) or a transformed cell such as a cancer cell.

T cells can be activated using antibodies directed against a number of cell surface antigens. These include components of the T cell receptor (TCR) such as the CD3 molecule, and other proteins including CD2, CD28, Thy-1, TAP (T cell activating protein) and Ly-6 (e.g. Ly-6C). Thus the antigen binding site of the antibody may be specific for CD3, CD2, CD28, Thy-1, TAP or Ly-6.

Preferably the antibody is specifically directed against the form of the relevant antigen from the species which it is intended to treat. In preferred embodiments, for treatment of humans, the antibody is directed against the human form of the relevant protein.

Antibodies against human proteins are typically derived from non-human species, and consequently their administration to humans may provoke an undesirable immune response against the antibody itself. It is possible to reduce immunnogenicity by making chimeric antibodies, which contain constant regions from human antibodies fused to the variable regions from the non-human antibody. A more sophisticated approach to “humanise” antibodies involves grafting the CDRs from the non-human antibody to human antibody framework regions. It is also now possible to generate fully human antibodies or fragments thereof by in vitro synthesis and screening (e.g. by phage display) or by producing antibodies from anials (e.g. mice) transgenic for human antibody genes.

Thus the antibody may comprise human constant regions and variable regions from a non-human antibody. Alternatively, it may comprise human framework regions, possibly with CDRs from a nonhuman antibody, e.g. one or more CDRs from OKT3 or UCHT-1, e.g. one, two, three, four, five or all the CDRs from OKT3 or UCHT-1. The CDR sequences for OKT3 are provided in U.S. Pat. No. 6,750,325.

The antibody may be humanised OKT3 (e.g. as described in U.S. Pat. No. 6,750,325) or humanised UCHT-1.

The antibody may comprise two Fab regions and a Fc region. Alternatively it may comprise or consist of a single chain Fv region, a Fab fragment, a Fab′ fragment or a F(ab′)₂ fragment.

It may be desirable for the antibody to possess at least two antigen binding sites (e.g. two Fab regions) specific for the cognate antigen on the immune cell surface. This allows the antibody to cross-link the antigen on the cell surface, which (depending on the identity of the antigen, and the specific antibody) may be necessary for cell activation, or may enhance cell activation. When the antibody possesses only one such antigen binding site, it preferably comprises an Fc region.

The antigen binding site will typically be inhibited from binding to the cell of the immune system by the presence of one or more blocking moieties coupled to the antibody via a selectively cleavable group or bond. The blocking moiety may be coupled at or adjacent the antigen binding site, and may sterically prevent binding between the antibody and its cognate epitope on the cell of the immune system.

The selectively cleavable group or bond may be cleavable by irradiation. Any frequency of radiation may be suitable, including infra-red (UV) radiation, visible light, ultra-violet (UV) radiation, microwaves, gamma rays, etc. depending on the type of blocking moiety. UV radiation is particularly convenient. The selectively cleavable group or bond may comprise a 1-(2-nitrophenyl)ethyl (NPE) moiety, which is cleavable by Uv radiation.

The antibodies described in this specification are useful for treating any condition which can be improved by selective activation of the immune system at a particular physiological site. Examples include cancers, particularly solid tumours, including ovarian cancer, mesothelioma, pancreatic cancer, melanoma, sarcoma, hepatoma, colon cancer, lung cancer, renal cancer, breast cancer, testicular cancer, prostate cancer. Other examples include infectious diseases, especially those in which a localised site of infection can be identified. The infectious agent responsible may be intracellular or extracellular, and may be bacterial, viral, fungal or any other type of infectious parasitic and/or pathogenic organism. The immune response may be directed against the infectious organism itself, or against host cells infected by the infectious organism.

The antibody may be administered directly to the physiological site where stimulation of the immune response is required. Alternatively, the antibody may be administered remote from the physiological site where stimulation of the immune response is required.

In either case, the resulting immune stimulation is restricted to the required site by providing localised activation of the antibody's binding capability. For example, in those embodiments where the antibody is inhibited by a photocleavable moiety, activation is typically achieved by irradiation at the physiological site where stimulation of the immune response is required.

The Examples show that an antibody inhibited by a photocleavable moiety not only provides significant therapeutic benefit when reactivated by irradiation, but actually provides greater benefit than an unmodified version of the same antibody.

Those experiments used a suspension of a particularly metastatic cell line, administered subcutaneously. The reversibly inhibited antibody was irradiated (and hence activated) only at the region where the tumour cells were administered. It might therefore have been expected that some cells would escape to form metastases elsewhere in the body, particularly in the liver. However no metastases were observed.

The antibody is also capable of providing benefit when irradiated ex vivo immediately before administration. Therefore in some embodiments it maybe desirable to activate the antibody's binding capability ex vivo immediately before administration to the subject, although this is not presently preferred.

In another aspect, the invention provides an antibody having an antigen binding site specific for CD3 and capable of activating a T cell on binding to CD3, said antibody being reversibly inhibited from binding to CD3 by the presence of at least one photocleavable moiety.

In another aspect, the invention provides a method of stimulating an immune response in a subject, comprising administering to said subject an effective amount of an antibody,
wherein the antibody comprises an antigen binding site capable of binding to a cell of the immune system such that binding of said antibody to said cell of the immune system activates said cell of the immune system,
wherein said antigen binding site is reversibly inhibited from binding to said cell of the immune system,
and wherein the antibody does not comprise a targeting moiety capable of binding to a cell which is not said cell of the immune system;
   the method further comprising restoring the ability of said antigen binding site to bind said cell of the immune system, thereby activating said cell of the immune system.

In another aspect, the invention provides an antibody for stimulating an immune response,

wherein the antibody comprises an antigen binding site capable of binding to a cell of the immune system such that binding of said antibody to said cell of the immune system activates said cell of the immune system,
wherein said antigen binding site is reversibly inhibited from binding to said cell of the immune system,
and wherein the antibody does not comprise a targeting moiety capable of binding to a cell which is not said cell of the immune system.

In another aspect, the invention provides the use of an antibody in the preparation of a medicament for stimulating an immune response against a target cell,

wherein the antibody comprises an antigen binding site capable of binding to a cell of the immune system such that binding of said antibody to said cell of the immune system activates said cell of the immune system,
wherein said antigen binding site is reversibly inhibited from binding to said cell of the immune system,
and wherein the antibody does not bind to the target cell.

In another aspect, the invention provides an antibody for stimulating an immune response against a target cell,

wherein the antibody comprises an antigen binding site capable of binding to a cell of the immune system such that binding of said antibody to said cell of the immune system activates said cell of the immune system,
wherein said antigen binding site is reversibly inhibited from binding to said cell of the immune system,
and wherein the antibody does not bind to the target cell.

In another aspect, the invention provides a method of stimulating an immune response against a target cell in a subject, comprising administering to said subject an effective amount of an antibody,
wherein the antibody comprises an antigen binding site capable of binding to a cell of the immune system such that binding of said antibody to said cell of the immune system activates said cell of the immune system,
wherein said antigen binding site is reversibly inhibited from binding to said cell of the immune system,
and wherein the antibody does not bind to the target cell,
   the method further comprising restoring the ability of said antigen binding site to bind said cell of the immune system, thereby activating said cell of the immune system.

In a further aspect, the invention provides a pharmaceutical composition comprising an antibody as described above in relation to any of the preceding aspects of the invention, in combination with a pharmaceutically acceptable carrier.

Preferred features of these aspects of the invention are as described above in relation to the first aspect.

### DESCRIPTION OF THE DRAWINGS

FIG. 1: Binding of OKT3 antibody to H9 T-cells as measured using FACS.

a, Control cells alone. b, T-cells+OKT3 antibody. c, T-cells+NPE-coated OKT3. d, T-cells+UV-A irradiated NPE-coated OKT3.

FIG. 2: Activation of T-cells as shown using a FITC-antiCD69 conjugate and FACS.

a, T-cells alone. b, T-cells and OKT3. c, T-cells with NPE-OKT3 added after irradiation of the cells. d, Irradiated T-cells and NPE-OKT3.

### DETAILED DESCRIPTION OF THE INVENTION

The antibody used in the compositions and methods of the invention has an antigen binding site capable of binding to a cell of the immune system and consequently activating the cell of the immune system. Thus, typically, the antibody is specific for a marker expressed on the surface of the immune cell. The marker is typically a protein. Cells of the immune system are typically activated by receiving signals from the environment (e.g. by binding of antigen to surface-expressed antibody molecules in B cells, or by binding of pro-inflammatory cytokines to specific receptors on the cell surface) or by direct interaction with other cells (e.g. by ligation of the T cell receptor by an MHC-antigen complex or homologue expressed on the surface of an antigen presenting cell). Such interactions may be mimicked by certain “agonist” antibodies, which are similarly capable of activating a cell of the immune system.

Activation of the cell of the immune system may cause it to secrete cytokines, proliferate, up- or downregulate expression of particular cell surface proteins (often referred to as “markers”) or display other behaviour characteristic of participation in an immune response at a level above its resting or background level. Thus the cell, once activated, will participate in mechanisms which initiate an immune response or enhance a pre-existing immune response at the relevant physiological site.

For example, T cells are lymphocytes which respond to antigen presented on the surface of other cells of the body in the context of specialised antigen presenting molecules. These antigen presenting molecules include MHC class I and II molecules, as well as others such as CD1. Most T cells (normally expressing the αβ T cell receptor) respond to peptide antigens presented by MHC molecules, but others (often expressing the γδ T cell receptor) respond to non-peptide antigens such as lipids.

The T cell receptor is expressed on the surface of the T cell and forms part of a complex containing CD3, which itself comprises at least 5 different polypeptide chains, designated γ, δ, ε, and ζ. One CD3 complex comprises one γ chain, one δ chain, and two of each of ε and ζ. Engagement of the T cell receptor by a suitable antigen presenting complex on the surface of another cell induces signalling via the CD3 complex which results in activation of the T cell.

A variety of different T cell subtypes exist. “Helper” T cells (which typically express CD4 and can themselves be divided into other subtypes including Th1 and Th2), when activated, proliferate and secrete cytokines to promote activation and proliferation of other immune cell types. “Cytotoxic” T cells (which typically express CD8), when activated, are capable of killing cells presenting the antigen to which they respond (which are often cancer cells and/or cells infected by parasites such as viruses).

It is well-known that antibodies against CD3 are capable of activating and inducing proliferation of T cells. Examples of anti-CD3 antibodies include OKT3 and UCHT-1, which are both directed against human CD3.

Activation of T cells may also be achieved using antibodies against Thy-1 (AAB26353.2 GI:13195770), TAP, Ly-6C, CD2 (NP_{—}001756.1 GI:4502653) and CD28 (NP_{—}006130.1 G1:5453611) (Takada, S, Engleman, E G, J. Immunol. 139:3231 (1987); Langlet, C, Guimezanes, A, Kaldy, P, et al. Cytotoxic T cells: Biology and Relevance to Diseases. eds. Battisto et al. Ann, N.Y. Acad. Sci. 532:33 (1988); Gunther, K C, Malek, T R, Shenvach, E M, J. Exp. Med. 159:716 (1984); Leo, O, Foo, M, Henkart, P A, et al. J. Immunol. 139:3556 (1987)).

To induce optimal stimulation via CD2, it may be desirable to use 2 or more different antibodies each binding to a different epitope on CD2. Known combinations include the T11.3 with either T11.1 or T11.2 antibodies (Meuer, S. C. et al. (1984) Cell 36:897-906) and the 9.6 antibody (which recognizes the same epitope as T11.1) in combination with the 9-1 antibody (Yang, S. Y. et al. (1986) J. Immunol. 137:1097-1100).

Alternatively, antibodies against one or more of these proteins may be used in conjunction with an antibody against CD3 to provide costimulatory signals to the T cell and thus enhance T cell activation. Costimulatory antibodies may be reversibly inhibited or not, as desired. When reversibly inhibited, it may be desirable that they are inhibited by the same mechanism as the anti-CD3 antibodies, so that one stimulus can activate both antibodies.

Characteristics of T cell activation depend on the cell subtype, but may include increased cell proliferation, increased IL-2 receptor expression, enhanced proliferation in response to IL-2 and increased expression of CD69. Helper T cells may secrete increased amounts of pro-inflammatory cytokines (e.g. IL-2, IFN-γ, TNFβ, IL-4, IL-5, IL-6, IL-10, GM-CSF, IL-10 and/or TNF-α, depending on the particular helper cell type), while cytotoxic T cells will display enhanced cytotoxic (cell-killing) activity. Thus antibody-induced activation of a T cell mimics antigen-specific activation of a T cell via the T cell receptor interaction, and induces qualitatively and/or quantitatively similar responses.

Antibodies against human CD3 include 7D6, 12F6, 38.1, 89b1, 131F26, BL-A8, BW239/347, BW264/56, CD3-4B5, CLB-T3/3, CRIS-7, F111-409, G19-4.1, HIT3a, ICO-90, IP30, Leu-4, LY17.2G3, M-T301, M-T302, MEM-57, MEM-92, NU-T3, OKT3 (U.S. Pat. No. 4,658,019), OKT3D, SMC2, T3, T3 (2Ad2), T3/2Ad2A2, T3/2AD, T3 (2ADA), T3/2T8-2F4, T3/RW2-4B6, T3/RW2-8C8, T10B9, T101-01, UCHT1, VIT3, VIT3b, X35-3, XXI11.46, XXI11.87, XXI11.141, YTH12.5, and YTH12.5. See the Human Leucocyte Differentiation Antigens (HLDA) Antibody Database for more details.

It is believed that antibodies having IgG2a isotype may be more effective at cell activation than antibodies of other IgG isotypes. Therefore the antibody may be IgG2a, particularly if it is an anti-CD3 antibody such as OKT3 or UCHT1.

The antibody may have the complete native antibody structure, consisting of two complete heavy chains linked by disulphide bonds to two complete light chains, appropriately folded to form two Fab regions and a Fc region, optionally with glycosylation. However it is well known that fragments of a whole antibody can perform the function of binding antigens. The term “antibody” is therefore used herein to encompass any molecule comprising the antigen binding portion of an antibody. Examples of binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward, E. S. et al., Nature 341, 544-546 (1989)) which consists of a VH domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding member (Bird et al, Science, 242, 423-426, 1988; Huston et al, PNAS USA, 85, 5879-5883, 1988).

The antibodies described herein possess at least one antigen binding site capable of binding to a cell of the immune system. It may be desirable that they comprise two (or more) such antigen binding domains. This may facilitate cross-linking of the cognate antigen on the surface of the cell of the immune system, which may be necessary (or optimal) for cell activation.

If the antibody possesses more than one antigen binding site, then all antigen binding sites are capable of binding to the same cell of the immune system, preferably to the same antigen on the cell of the immune system, and preferably to the same epitope on that antigen. For example, all antigen binding sites of the antibody may be identical.

However, activation of some immune cell types may require ligation of more than one type of cell surface receptor. This may be achieved by co-administration of two different types of antibody as described in this specification, each antibody being specific for one of the receptors on the immune cell. Alternatively, an antibody may be used which comprises two different antigen binding sites, each being specific for a different receptor on the surface of the immune cell; i.e. the antibody may be bispecific. For example, it may bind to both CD3 and CD28.

The antibody does not possess an antigen binding site capable of binding to a cell type other than the cell of the immune system which is to be activated. Furthermore the antibody has not been modified or engineered to carry any other targeting moiety which targets the antibody to another cell type (i.e. causes the antibody to bind to that cell at a level significantly above background binding of the same antibody lacking the targeting moiety). The antibody has not been modified or engineered to carry any non-antibody moiety, excluding a blocking moiety which may be present as discussed further below. Thus it has not been engineered to carry ligands or receptors for molecules expressed on other cell types. It is, of course, appreciated that antibody Fc regions are capable of binding to Fc receptors expressed on various cell types. However, the Fc region is not considered here to be a targeting moiety which has been deliberately and artificially introduced to the antibody in order to enable it to bind another cell type. Neither is an antibody's natural glycosylation considered to be such a targeting moiety. Thus, preferably, the antibody binds above background levels only to cells expressing the cognate antigen for the antigen binding site, and (if the antibody comprises an Fc domain) to cells expressing Fc receptors.

In particular, where the antibody is to be used to stimulate an immune response against a target antigen or a target cell type, the antibody is not capable of binding to that target antigen or target cell type, either via an antigen binding site or via a heterologous moiety introduced by artificial means (e.g. genetic engineering or chemical modification).

The antibody is reversibly inhibited from binding to its cognate antigen. Typically this is achieved by chemical conjugation of a blocking moiety, which can be selectively removed in order to activate the antibody. Thus a blocking moiety may be linked to the antibody by a selectively cleavable group or bond.

In preferred embodiments the selectively cleavable group or bond is cleavable by irradiation, e.g. by UV, infra-red, X-ray or visible irradiation. Laser irradiation may be particularly suitable, especially for therapeutic methods, as its delivery can be very closely controlled. Thus it can be used to irradiate only a site of diseased tissue (e.g. a tumour) without affecting surrounding healthy tissue. However irradiation from a UV lamp may be equally suitable. It may be possible to activate the antibody's binding capability by irradiation through the recipient's skin, so avoiding the need for surgical intervention.

Thus the antibody may be inhibited from binding to its cognate antigen by a photocleavable moiety. Such photocleavable moieties are well known in the art. Antibodies can be suitably derivatised by means of appropriate reagents which couple to hydroxy or amino residues. Thus phosgene, diphosgene or DCCl (dicyclohexyl carbodiimide) may be used to generate photocleavable esters, amides, carbonates and the like from a wide variety of alcohols. Nitrophenyl derivatives may be used in this context. Substituted arylalkanols may be used, such as nitrophenyl methyl alcohol, 1-nitrophenylethan-1-ol, and substituted analogues. Thompson et al. (Biochem. Biophys. Res. Com. 201, 1213-1219 (1994) and Biochem. Soc. Trans. 225S, 23 (1995)) describe reversible inhibition of protein function by addition of 1-(2-nitrophenyl)-ethyl (NPE) moieties. Further photocleavable moieties will be well known to the skilled person, e.g. from “Biological Applications of Photochemical Switches”, H. Morrison (ed.), Bioorganic Photochemistry Series, Volume 2, J. Wiley & Sons. (see especially Chapter 1, section 4, pages 34 to 50). Other suitable photocleavable moieties include 1-(2-nitrophenyl)diazoethane (L. Bédouet et al., Recovery of the oxidative activity of caged bovine haemoglobin after UV photolysis, BBRC, 320 (2004) 939-944), 2-nitrophenylglycine (M. Endo et al, Design and synthesis of photochemically controllable caspase-3, Angew. Chem. Int. Ed 2004, 43, 5643-5645), 6-nitroveratryl (M. Endo et al, Design and synthesis of photochemically controllable restriction endonumclease BamHI by manipulation of the salt-bridge network in the dimmer interface, J. Org. Chem., 2004, 69, 4292-4289), o-nitrobenzyl and 4-hydroxyphenacyl.

The antibodies described in this specification will typically be administered to a recipient in the form of pharmaceutical compositions. These compositions may comprise, in addition to the antibody, a pharmaceutically acceptable excipient, carrier, buffer, stabiliser or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, intraperitoneal routes or topical application. Intravenous, intramuscular or subcutaneous administration is likely to be appropriate in many instances, but other methods of administration are possible.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection (which may or may not be at the site where immune stimulation is desired), the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabililsers, buffers, antioxidants and/or other additives may be included, as required.

Administration is preferably in an “effective amount” sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Suitable carriers, adjuvants, excipients, etc. can be found in standard pharmaceutical texts, for example Remington's Pharmaceutical Sciences, 20th Edition, 2000, pub. Lippincott, Williams & Wilkins; and Handbook of Pharmaceutical Excipients, 2nd edition, 1994. However the dose administered will be suitable to stimulate immune cells rather than suppress immune responses. In general lower doses of anti-CD3 are believed to stimulate T cells, while higher doses are more likely to suppress immune responses, possibly by depletion of T cells. See Bluestone, J A, Science 242:569 (1988), and U.S. Pat. No. 6,406,696. The skilled person will be capable of ascertaining a suitable dose for any given situation.

The compositions and methods described herein are preferably used for treatment of mammals, more preferably primates (e.g. humans, apes or monkeys), domestic animals (e.g. feline, canine, etc.), laboratory animals (e.g. rodents, lagomorphs etc.) or livestock animals (e.g. bovine, equine, porcine, etc.).

### EXAMPLES

### Methods

### Antibodies and Cell Lines

The CD3+T-cell line H9 was obtained from ETCC. The OKT3 secreting hybridoma was obtained from ATCC and OKT3 was obtained from serum free media by (NH₄)₂SO₄ precipitation. The human ovarian cell line, OAW42, which was used to produce conditioned RPMI media was kindly provided by Dr A Wilson, Derby General Hospital.

The CD3+ murine cell line EL4 was stored in this laboratory. This cell line was grown in RPMI 1640 media containing 10% FCS. The 145-2C11 monoclonal antibody secreting hybridoma was obtained from ECACC. The antibody was precipitated from serum free media using (NH₄)₂SO₄ followed by extensive dialysis.

### Reversible Inhibition of Antibodies

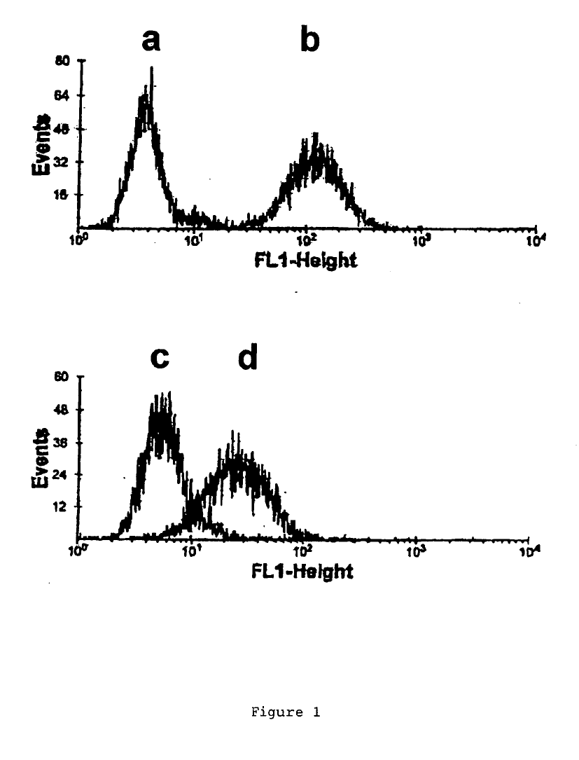

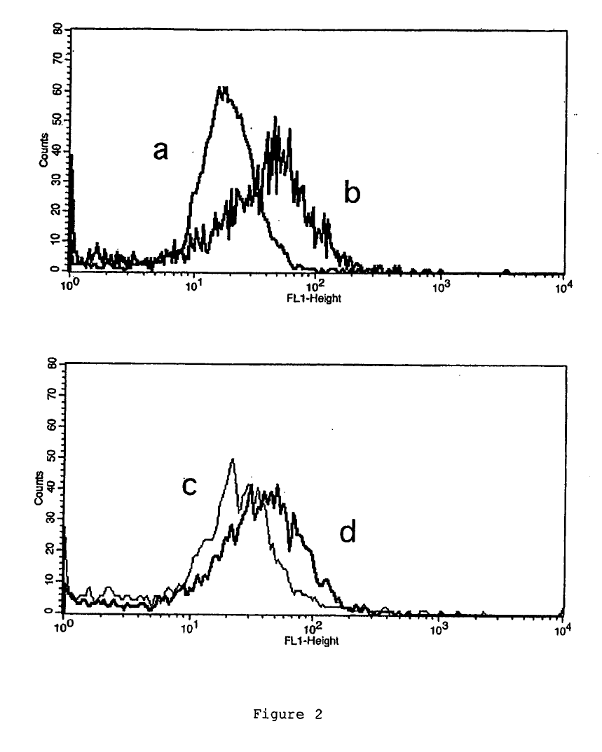

OKT3 (0.5 mg in 1 ml 0.1M Bicarbonate) was rendered inactive by the addition of 15 μl NPE-carbonyl chloride (in dioxan) for 4 h at 20° C. followed by centrifugation and dialysis to remove excess reagents and insoluble reaction products⁸. When this was irradiated with UV-A light the NPE groups cleaved, reactivating the antibody. In early experiments the NPE-coated OKT3 was irradiated in a Quartz cuvette prior to its addition to the T-dells (FIG. 1). In later work the NPE-OKT3 complexes were irradiated in the presence of the T-cells (FIG. 2) in 96 well plates.

Likewise, 1.5 ml of 145-2C11 (0.5 mg/ml in 0.1M Bicarbonate) was inactived by the addition of 10 μl NPE-carbonyl chloride (in dioxan) for 4 h at 20° C. followed by repeated dialysis in 10 mM Phosphate buffer pH7.5 to remove uncoupled NPE groups (1). On centrifugation at 13,000 g for 10 min the inactivated 145-2C11 was obtained as a pellet. This was resuspended in 1 ml of Phosphate buffered saline to give a suspension containing 0.19 mg/ml inactivated antibody with an 01)₂₈₀ value of 1.4. After subtracting the OD₂₈₀ value for the antibody (0.26) the remaining OD of 1.14 equates with approx. 300 NPE residues being associated on each antibody molecule. This figure is almost certainly far too high due to the NPE-coated antibody being a cloudy suspension and reflecting rather than absorbing the light beam. When this suspension was irradiated with UV-A light the NPE groups cleaved, reactivating the antibody.

### Photolysis of Conjugates in Vitro.

The NPE-coated OKT3 samples were irradiated with a VL-206BL UV-A lamp (2×6W tubes) which has a total UV-A irradiance of approx. 16 mW/cm² at a distance of 1 cm. Irradiations were carried out for six minutes in quartz cuvettes in preliminary experiments which demonstrated that >90% of the NPE residues cleave in this time with no denaturing of antibody activity. Longer periods of UV-irradiation (10 min) were used when the cells and NPE complexes were irradiated together to allow for absorption of the UV light by the plastic lid of the 96 well plates.

### Binding of OKT3 Antibody to H9 T-cells by FACS Analysis

250 ul aliquots of H9 cells, 10⁶ cells/ml in RPMI 1640 media containing 10% FCS, had 30 ul of untreated or NPE-coated OKT3(0.1 mg/ml) added to them and were left for 1 h at 4° C. After washing the cells were resuspended in 100 ul of FITC-labelled goat anti-mouse (BD Pharmingen, 5 ul diluted to 1 ml in phosphate buffered saline pH 7.4, PBS) and left for 30 min at 4° C. After 3 further washes the cells were resuspended in PBS and OKT3 binding was analysed by FACS.

### Expression of CD69 and IL2 by H9 Cells

The expression of the activation marker CD69 was analysed using FACS. The H9 human T-cells were resuspended in OAW conditioned RPMI medium, 150 ul aliquots containing 150,000 T-cells were added to individual wells of a 96 well plate. 20 ul of untreated or NPE-coated OKT3 (0.2 mg/ml) was added to the wells and the cells were irradiated in the presence of the antibody for 10 min through the lid of the plate. Unirradiated NPE-coated OKT3 was then added to relevant wells and the cells were left for 3 h at 37° C. before the supernatant was removed (for IL2 analysis). After washing the cells were resuspended in 50 ul PBS containing FITC-labelled anti-CD 69 (5 ul per 10⁶ cells) and were left for 1 hr at 4° C. After 3 further washes the cells were resuspended in PBS and analysed immediately using FACS. IL2 concentrations were measured using a BD Biosciences human IL2 ELISA kit. OAW conditioned medium (RPMI 1640 media containing 10% FCS which had been left for 3 days in confluent cultures of OAW42 cells) was required for the H9 cells to express CD69 and IL2.

### Tumour Growth

C57BL6 mice were purchased at 8 weeks old and were injected with tumour after they had been left for at least one week to acclimatise to their new surroundings. Frozen pieces of M5076 tumour were thawed from liquid nitrogen storage. This was diced as finely as possible in 199 medium and 50 ul of packed diced tumour was injected subcutaneously into each animal using a fine guage needle. After approx 3 weeks the tumours were excised and freshly diced tumour (50 ul) was simultaneously injected with 50 ul of medium containing 5 ug of 145-2C11 conjugates. Controls contained medium alone.

For in vivo photolysis a small area on the flank of the mice was shaved using hair clippers, the tumour and antibody were injected under the shaven area, and the shaven area was irradiated for 5 min with a hand held lamp (see below) from a distance of 2-3 cm above the mice.

### Results

### Reversible Inhibition of OKT3 and Activation of T Cells In Vitro

A coating of photocleavable 2-(nitrophenyl)ethanol (NPE) groups was used to inhibit the biological activity of the anti-CD3 monoclonal antibody OKT3. The inhibition and reactivation of OKT3 binding to the H9 human CD3+cell line was then investigated using FACS. Native uncoated antibody was taken as representing 100% binding (FIG. 1b). When the antibody was coated with NPE its binding to the T-cell line reduced markedly with only 2.5% of the cells fluorescing (FIG. 1c), a figure similar to that given by control cells incubated with only the second layer antibody (FIG. 1a). Irradiation with UV-A light removed the NPE groups and reactivated the antibody as shown by the antibody binding increasing to approximately 80% of that given by untreated native OKT3 (FIG. 1d).

The light specific binding and subsequent activation of the H9 T-cell line was confirmed by the expression of early T-cell activation markers 3 h after the addition of the NPE-OKT3 complexes. In T-cells treated with un-illuminated NPE-OKT3 the levels of activation marker CD69 (FIG. 2c) were only slightly increased above background fluorescence (FIG. 2a). However the CD69 levels were significantly increased on the T-cells illuminated in the presence of NPE-OKT3 (FIG. 2d) to levels similar to those obtained with control illuminated uncoated antibody (FIG. 2b). This light controlled activation of the H9 cells was confirmed by the analysis of IL-2 levels in the cell culture supernatants. Medium from control H9 cells which did not receive cloaked antibody contained 11±3 pg/ml IL2. In medium from cells treated with NPE-OKT3 the concentration of IL2 increased to 54±13 pg/ml IL2. (This was not unexpected and probably reflected the presence of a small residual fraction of uncloaked antibody). However, illumination of the H9 cell NPE-OKT3 mixture increased the IL2 concentration to 211±30 pg/ml (Mean and variation of 3 separate experiments).

It is important here to point out that i) reactivation was carried out in the presence of the H9 cells under physiological conditions without causing damage to the cells and ii) the light from the hand held lamp was sufficient to reactivate the OKT3 antibody even though the irradiation was carried out through the plastic lid of the 96 well plate.

We have also been able to demonstrate the reversible inhibition of UCHT-1, a second anti-human CD3 antibody, with very similar results to those reported above. This establishes the reproducibility of the procedure described.

### Reversible Inhibition of 145-2C11

As we had successfully reversibly inactivated the mouse anti-human T-cell antibody, OKT3, we then decided to examine if we could reproduce this effect with the similar hamster anti-mouse CD3 antibody, 145-2C11 (20). This would enable us to investigate possible anti-cancer effects of our procedure in a syngeneic C57BL6 mouse system. The 145-2C11 antibody was therefore coated with NPE. This antibody proved to be prone to coming out of solution during dialysis to remove excess NPE. When resuspended to a suspension in isotonic saline the NPE-coated 145-2C11 antibody could not bind to the CD3 expressing murine lymphoma cell line EL4. However after illumination with UV light for 10 min considerable binding of the antibody (70% of that given by uncoated antibody) to EL4 cells could be detected (Table 1). This was an extremely important result, as this was carried out in the presence of the cells under physiological conditions.

### Inhibition of Tumour Growth In Vivo

We then employed the NPE-coated 145-2C11 antibody to carry out some initial studies to determine if we could regulate the growth of the M5076 ovarian tumour cell line in C57BL6 mice. Initial toxicology studies demonstrated high doses of 145-2C11 and NPE-coated 145-2C11 (30 ug/mouse) had no deleterious effects on the mice. We grew up the M5076 ovarian tumour in C57BL6 mice, finely diced the resulting tumour and passaged 50 ul of the diced tumour into groups of 6 mice along with 50 ul of medium containing 5 ug of various 145-2C11 conjugates. After approximately 3 weeks the mice were humanely killed and the final tumour weights were measured. The results obtained from two separate experiments are given in Table 2.

In the first experiment the control tumours had an average weight of 200 mg in the mice that only received media with the transplanted diced tumour. When control uncoated 145-2C11 was injected with the diced tumour, final tumour weights markedly reduced to around 20 mg. This is an interesting finding in itself, as it shows that if an anti-CD3 antibody is present in the area next to a tumour it will stimulate the immune response, even when it is not being specifically targeted to the tumour by a bi-specific antibody. When the NPE-coated-inactivated antibody was injected with the diced tumour the average weight of the final tumours increased in size almost back to the level of control tumours (as expected). If the NPE-coated 145-2C11 was irradiated in vitro for 5 min (in a cuvette) then injected with the transplanted tumour, then the final tumour weight decreased significantly, but not to the levels found with uncoated antibody. However when the tumour and NPE-coated 145-2C11 was irradiated in vivo after the two components had been injected, there was a massive drop in subsequent tumour growth to the extent that no tumour was detectable in 5 of the 6 mice after 25 days. This was even more pronounced than the effect of uncoated antibody. This implied that irradiation of the NPE-coated 145-2C11 in situ with the tumour conferred some additional potency.

In order to confirm these results the experiment was repeated. Tumour growth was more vigorous on this passage of tumour, control tumours and tumours treated with unirradiated inactivated 145-2C11 reaching 400 mg in size after only 22 days growth. Treatment with uncoated antibody again markedly decreased tumour growth to around 25% of control values whilst in vitro irradiated NPE-coated antibody again reduced final tumour sizes to just over half control values. Despite the more vigorous growth of the tumour in this experiment, very little tumour was again obtained in the animals that were irradiated in vivo to reactivate the 145-2C11.

A third experiment was then carried out in which control tumours were irradiated through a patch of shaved skin in addition to the animals treated with NPE inactivated 145-2C11 (Table 3). Here the tumours again grew more vigorously to a very similar size (365 mg) obtained in the previous experiment. The irradiated control tumours were no smaller than un-irradiated control tumours confirming that reductions in tumour growth were caused by the reactivated 145-2C11 antibody. The tumours in the latter group, whose transplant had been irradiated in the presence of NPE-coated antibody, were barely detectable. Older mice had been used for this experiment and it was very hard to distinguish and dissect the tiny tumours from surrounding fat. Figures given for the final tumour weights in this group are over-estimates due to fat contamination.

TABLE 1
The binding of 145-2C11 control and
NPE coated conjugates to EL4 cells.

| 145-2C11-Conjugate | Mean |
|---|---|
| added | Fluorescence |
| None | 12 |
| 145-2C11 (stock) | 59 |
| 145-2C11 (control | 56 |
| after going through | |
| whole coating procedure | |
| but no NPE added) | |
| NPE-145-2C11 | 13 |
| NPE-145-2C11 + UV | 42 |

100 ul aliquots containing 200,000 EL4 cells were added to individual wells of a 96 well plate. 10 ul of NPE-coated 145-2C11 (0.19 mg/ml) was added to the wells and the cells were irradiated in the presence of the antibody for 10 min through the lid of the plate. Unirradiated NPE-coated 145-2C11 was then added to relevant wells and the cells were left for 1 h at 4° C. before they were washed. After washing the cells were resuspended in 100 ul of a 1/100 dilution of Biotinylated-anti hamster IgG (Vector labs) for 30 min, washed, and 100 ul of a 1/1000 dilution of Strepavidin-FITC (Sigma) was added, again for 30 min at 4° C. After 3 further washes the cells were resuspended in PBS and analysed immediately using FACS. The value given is the mean fluorescence of the FACS peak.

TABLE 2
M5076 tumour weights obtained in mice treated with
various 145-2C11 conjugates

| | | Control | | | | | | | |
|---|---|---|---|---|---|---|---|---|---|
| Medium | | uncoated | | NPE-coated | | NPE-coated | | NPE-coated | |
| control | | Antibody | | UV in vitro | | no UV | | UV in vivo | |
| 0.06 | Av wt | 0 | Av wt | 0.05 | Av wt | 0.08 | Av wt | 0.07 | Av wt |
| 0.27 | 200 mg | <0.01 | 20 mg | 0.12 | 110 mg | 0.19 | 160 mg | 0 | 10 mg |
| 0.18 | | <0.01 | | 0.18 | | 0.24 | | 0 | but No Trace |
| 0.08 | | 0.02 | | 0.18 | | 0.19 | | 0 | in 5 |
| 0.40 | | 0.13 | | 0.03 | | 0.13 | | 0 | mice |
| 0.18 | | <0.01 | | 0.08 | | 0.13 | | 0 | |
| 0.03 | Av wt | 0.09 | Av wt | 0.14 | Av wt | 0.53 | Av wt | 0.04 | Av wt |
| 0.47 | 380 mg | 0.12 | 110 mg | 0.46 | 270 mg | 0.37 | 400 mg | 0.03 | 30 mg |
| 0.46 | | 0.20 | | 0.14 | | 0.25 | | 0.05 | |
| 0.55 | | 0.10 | | 0.29 | | 0.32 | | <0.01 | |
| 0.31 | | 0.12 | | 0.24 | | 0.56 | | 0 | |
| 0.46 | | 0.05 | | 0.34 | | 0.37 | | 0.07 | |

Groups of 6 mice were simultaneously injected (subcutaneously) with 50 ul of diced M5076 tumour and 50 ul of medium containing 145-2C11 conjugates. After approx. 3 weeks the resulting subcutaneous tumours were excised and weighed. Values are given (in grams) for each animal. Statistics analysis is not required as the differences in tumour weights are so large in the different groups. Data is given for two separate sets of experiments.

TABLE 3
M5076 tumour weights obtained in control mice compared
to mice treated with NPE-coated 145-2C11.

| | | |
|---|---|---|
| Medium | Medium | NPE-coated |
| control | Control | 145-2C11 |
| No UV | UV in vivo | UV in vivo |

| | | | | | |
|---|---|---|---|---|---|
| *0.17 | Av wt | 0.37 | Av wt | <0.04 | Av wt |
| *0.11 | >210 mg | 0.50 | 365 mg | <0.03 | <55 mg |
| 0.30 | | 0.29 | | <0.07 | |
| 0.26 | | 0.30 | | <0.08 | |

Three groups of 4 mice were simultaneously injected with 50 ul of diced M5076 tumour and 50 ul of medium (2 groups) or NPE-coated 145-2C11 (1 group). One of the control groups and the NPE-coated antibody group were irradiated for 5 min through a shaved patch of skin. * Two tumours were not completely excised.

While the invention has been described in conjunction with the exemplary embodiments described above, many equivalent modifications and variations will be apparent to those skilled in the art when given this disclosure. Accordingly, the exemplary embodiments of the invention set forth are considered to be illustrative and not limiting. Various changes to the described embodiments may be made without departing from the spirit and scope of the invention. All references cited herein are expressly incorporated by reference.

### REFERENCES

1. Baeuerle P A, Kufer P, Lutterbuse R. Bispecific antibodies for polyclonal T-cell engagement. Curr Opin Mol Therapeut 2003; 5: 413-419.
2. Lum L G, Davol P A. Retargeting T cells and immune effector cells with bispecific antibodies. Cancer Chemother and Biological response modifiers 2005; 22: 273-291.
3. van Spriel A B, van Ojik H H, van de Winkel J G J. Immunotherapeutic perspective for bispecific antibodies. Immunology Today 2000; 21: 391-402.
4. Withoff S, Helfrich W, de Leij L F, Molema G. Bi-specific antibody therapy for the treatment of cancer. Curr Opin Mol Therapeut 2001; 3: 53-62
5. Morrow K J, Jr. Challenges remain for antibody products. Genet Engineering News 2005; 25: No 19 pages 1, 16-19.
6. Brekke O H, Sandlie I. Therapeutic antibodies for human diseases at the dawn of the twenty-first century, Nature Reviews 2002; 2: 52-62.
7. Molema G, Cohen Tervaert J W, Kroesen B J, Helfrich W, Meijer D K, de Leij L F. CD3 directed bispecific antibodies induce increased lymphocyte-endothelial cell interactions in vitro. Br J Cancer 2000; 82: 472-479.
8. Thompson S, Fawcett M-C, Spoors J A, Self, C H. The modulation of protein A-IgG(Fc) binding by the reversible addition of 2-nitrobenzyl groups. Biochem Soc Trans 1995; 23: 155S.
9. Self C H, Thompson S. Light activatable antibodies: Models for remotely activatable proteins. Nature Medicine 1996; 2: 817-820.
10. Kossel A H, Cambridge S B, Wagner U, Bonhoeffer T, A caged Ab reveals an immediate/instructive effect of BDNF during hippocampal synaptic potentiation, Proc Natl Acad Sci USA 2001; 98:14702-14707.
11. Goeldner M, Givens R (eds.), Dynamic studies in biology:Phototriggers, Photoswitches and Caged Biomolecules. Wiley-VCG, Weinheim, 2005.
12. Renner C, Pfreundschuh M. Tumor therapy by immune recruitment with bispecific antibodies. Immunol Revs 1995; 145: 179-209.
13. Porter L E, Nelson H, Ethem G I, Rice D C, Thibault C, Chapoval A I. T cell activation and retargeting using staphylococcal enterotoxin B and bispecific antibody: an effective in vivo antitumor strategy. Cancer Immunol Immunother 1997; 45: 180-183.
14. Pfosser A, Brandi M, Salih H, Grosse-Hovest L, Jung G. Role of target antigen in bispecific-antibody-mediated killing of human glioma cells: A preclinical study. Int. J. Cancer 1999; 80: 612-616.
15. Chapoval A I, Nelson H, Thibault C. Anti-CD3 x Anti tumour F(ab′)2 bifunctional antibody activates and retargets Tumor-Infiltrating Lymphocytes. J Immunol 1995; 155: 1296-1303.
16. Riedle S, Rosel M, Zoller, M. In Vivo Activation and expansion of T cells by a bi-specific antibody abolishes metastasis formation of human melanoma cells in scid mice. Int J Cancer 1998; 75: 908-918.
17. Rietz C, Chen L. New B7 family members with positive and negative costimulatory function. Am J Transplantion 2004; 4: 8-14.
18. da Costa L, Renner C, Hartmann E, Pfreundschuh, M. Immune recruitment by bispecific antibodies for the treatment of Hodgkin disease. Cancer Chemother & Pharmacol 2000; 46: S33-36.
19. Katzenwadel A, Scheer H, Gierschner D, Wetterauer U, Elsasser-Beile U. Construction and in vivo evaluation of an anti-PSA x anti-CD3 bispecific antibody for the immunotherapy of prostate cancer. Anticancer Res 2000; 20: 1551-1555.
20. Alegre M-L, et al. Cytokine release symdrome induced by the 145-2C11 anti-CD3 monoclonal antibody in mice: Prevention by high doses of methylprednisolone. J. Immunol. 146, 1184-1191, 1991.

1-19. (canceled)

20. An antibody or functional fragment thereof having an antigen binding site specific for CD3 and capable of stimulating a T cell on binding to CD3, said antibody being reversibly inhibited from binding to CD3 by the presence of at least one photocleavable moiety.

21. A method of stimulating an immune response in a subject, comprising administering to said subject an effective amount of an antibody or functional fragment thereof,
wherein the antibody or functional fragment thereof comprises an antigen binding site capable of binding to a cell of the immune system such that binding of said antibody to said cell of the immune system activates said cell of the immune system,
wherein said antigen binding site is reversibly inhibited from binding to said cell of the immune system,
and wherein the antibody or functional fragment thereof does not comprise a targeting moiety capable of binding to a cell which is not said cell of the immune system;
the method further comprising restoring the ability of said antigen binding site to bind said cell of the immune system, thereby activating said cell of the immune system.

22. An antibody or functional fragment thereof for stimulating an immune response, wherein the antibody comprises an antigen binding site capable of binding to a cell of the immune system such that binding of said antibody to said cell of the immune system activates said cell of the immune system, wherein said antigen binding site is reversibly inhibited from binding to said cell of the immune system, and wherein the antibody does not comprise a targeting moiety capable of binding to a cell which is not said cell of the immune system.

23. A pharmaceutical composition for stimulating an immune response against a target cell, comprising the antibody of claim 20 in a pharmaceutically acceptable carrier.

24. An antibody or functional fragment thereof for stimulating an immune response against a target cell, wherein the antibody comprises an antigen binding site capable of binding to a cell of the immune system such that binding of said antibody to said cell of the immune system activates said cell of the immune system, wherein said antigen binding site is reversibly inhibited from binding to said cell of the immune system, and wherein the antibody does not bind to the target cell.

25. A method of stimulating an immune response against a target cell in a subject, comprising administering to said subject an effective amount of an antibody,
wherein the antibody comprises an antigen binding site capable of binding to a cell of the immune system such that binding of said antibody to said cell of the immune system activates said cell of the immune system,
wherein said antigen binding site is reversibly inhibited from binding to said cell of the immune system, and
wherein the antibody does not bind to the target cell, the method further comprising restoring the ability of said antigen binding site to bind said cell of the immune system, thereby activating said cell of the immune system.

26. (canceled)

27. The method according to claim 21 wherein the cell of the immune system is a T cell.

28. The method according to claim 27, wherein the T cell is a cytotoxic T cell.

29. The method according to claim 27, wherein the antigen binding site is specific for a component of the T cell receptor (TCR).

30. The method as claimed in claim 29 wherein the antigen binding site is specific for CD3.

31. The method according to claim 30 wherein the antigen binding site comprises at least one CDR from OKT3 or UCHT-I.

32. The method according to claim 31 wherein the antigen binding site comprises all the CDRs from OKT3 or UCHT-I.

33. The method of claim 21 wherein the antibody has human constant regions or human framework regions.

34. The method as claimed in claim 33, wherein the antibody is humanised OKT3 or UCHT-I.

35. The method as claimed in claim 21, the functinal fragment of said antibody comprises a scFv, Fab, Fab′ or F(ab′)2.

36. The method of claim 21, wherein the binding of said antibody or functional fragment thereof to said cell of the immune system is reversibly inhibited by a blocking moiety which is coupled to the antibody via a selectively cleavable group or bond.

37. The method of claim 36, wherein the selectively cleavable group or bond is cleavable by irradiation.

38. The method of claim 37 wherein the irradiation is UV irradiation.

39. The method according to claim 36, wherein the selectively cleavable group or bond comprises a 1-(2-nitrophenyl)ethyl (NPE) moiety.

40. The method according to claim 21 for stimulating an immune response against a tumour.

41. The method according to claim 21 for stimulating an immune response against an infectious organism or a host cell infected by an infectious organism.

42. The method according to claim 21, wherein the antibody is administered to the physiological site where stimulation of the immune response is required.

43. The method according to claim 21, wherein the antibody is administered remote from the physiological site where stimulation of the immune response is required.

44. The method according to claim 21 wherein irradiation is applied at the physiological site where stimulation of the immune response is required.
